(19) United States
(12) Patent Application Publication
(10) Pub. No.: US 2020/0352667 A1
(43) Pub. Date: Nov. 12, 2020

(54) SURGICAL ROBOTIC ARMS AND PULLEY ASSEMBLIES THEREOF

(71) Applicant: Covidien LP, Mansfield, MA (US)

(72) Inventors: Jaimeen Kapadia, Cambridge, MA (US); Josiah Rosmarin, Sebring, FL (US)

(21) Appl. No.: 16/961,048

(22) PCT Filed: Jan. 9, 2019
(86) PCT No.: PCT/US2019/012839

§ 371 Date: Jul. 9, 2020

Related U.S. Application Data
(60) Provisional application No. 62/615,578, filed on Jan. 10, 2018.

(51) Int. Cl. A61B 34/00; A61B 34/37; A61B 90/00; B25J 9/10

(52) U.S. Cl. CPC A61B 34/71; A61B 34/37; A61B 2034/302; B25J 9/1045; A61B 90/03

(57) ABSTRACT

A pulley assembly of a robotic arm includes a first cable, a first pulley, and a second pulley. The first pulley has a first end of the first cable fixed thereto. The second pulley is operably coupled to the first pulley via the first cable. The second pulley includes a hub, a first semicircular body rotatably attached to the hub, and a first fastener movably coupled to the hub. The first semicircular body has a second end of the first cable fixed thereto. Movement of the first fastener rotates the first semicircular body relative to the hub to change a tension in the first cable.

### BACKGROUND

Robotic surgical systems have been used in minimally invasive medical procedures. Some robotic surgical systems included a console supporting a surgical robotic arm and a surgical instrument or at least one end effector (e.g., forceps or a grasping tool) mounted to the robotic arm. The robotic arm provided mechanical power to the surgical instrument for its operation and movement. Each robotic arm may have included an instrument drive unit operatively connected to the surgical instrument.

Robotic arms may be constructed of a plurality of joints that are movable relative to one another via a cable system. The cables may have been fabricated from stainless steel, which may result in stretching of the cables over time, thereby effecting operation of the robotic arm. Accordingly, a need exists to provide robotic arm cables that resist deformation over time, and a way to selectively tension the robotic arm cables during or prior to usage.

### SUMMARY

In one aspect of the present disclosure, a robotic arm is provided. The robotic arm includes a first elongate member, a second elongate member, a third elongate member, and motor, and a pulley assembly. The first elongate member has a first end configured to be coupled to a fixed surface, and a second end. The second elongate member has a first end rotatably connected to the second end of the first elongate member, and a second end. The third elongate member has a first end rotatably connected to the second end of the second elongate member, and a second end configured to be coupled to a surgical instrument. The motor is attached to the second end of the first elongate member and configured to rotate the second elongate member relative to the first elongate member. The pulley assembly is disposed within the second elongate member and includes a first pulley disposed within the second end of the second elongate member, and a second pulley operably coupled to the first pulley via a first cable. The second pulley includes a hub disposed within the first end of the second elongate member, a first semicircular body rotatably attached to the hub, and a first fastener movably coupled to the hub. Movement of the first fastener rotates the first semicircular body relative to the hub to change a tension in the first cable.

In some embodiments, the second pulley may include a second semicircular body rotatably attached to the hub of the second pulley, and a second fastener movably coupled to the hub of the second pulley such that movement of the second fastener rotates the second semicircular body relative to the hub of the second pulley to change a tension in a second cable. The first cable may have a first end fixed to the first semicircular body and a second end fixed to the first pulley, and the second cable may have a first end fixed to the second semicircular body and a second end fixed to the first pulley. The first and second semicircular bodies may be disposed adjacent one another.

It is contemplated that the first fastener may be a screw that is threadingly coupled to the hub of the second pulley. The screw may have an end in abutting engagement with the first semicircular body. Rotation of the screw may drive the end of the screw toward the first semicircular body to rotate the first semicircular body.

It is envisioned that the first semicircular body may have a first end and a second end and be disposed about the hub of the second pulley. The first fastener may have an end in abutting engagement with the first end of the first semicircular body. The first cable may have an end that is fixed to the second end of the first semicircular body.

In some embodiments, the first cable may have a first end fixed to the first pulley and a second end fixed to the first semicircular body such that rotation of the first semicircular body relative to the hub of the second pulley increases the tension in the first cable.

In another aspect of the present disclosure, a pulley assembly of a robotic arm is provided and includes a first cable, a first pulley, and a second pulley. The first pulley has a first end of the first cable fixed thereto. The second pulley is operably coupled to the first pulley via the first cable. The second pulley includes a hub, a first semicircular body, and a first fastener. The first semicircular body is rotatably attached to the hub and has a second end of the first cable fixed thereto. The first fastener is movably coupled to the hub. Movement of the first fastener rotates the first semicircular body relative to the hub to change a tension in the first cable.

Further details and aspects of exemplary embodiments of the present disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the present disclosure are described herein with reference to the accompanying drawings, wherein:

FIG. 1 is a schematic illustration of a robotic surgical system including a surgical robotic arm in accordance with the present disclosure;

FIG. 2 is a side, perspective view of the surgical robotic arm of FIG. 1;

FIG. 3 is an enlarged perspective view of the surgical robotic arm of FIG. 1, with parts removed.

FIG. 4 is a perspective view of a pulley assembly of the surgical robotic arm of FIG. 1;

FIG. 5 is an enlarged, perspective view of the pulley assembly of FIG. 4;

FIG. 6A is an enlarged, side view of the pulley assembly of FIG. 4;

FIG. 6B is an enlarged, side view of the pulley assembly of FIG. 4;

FIG. 7 is a perspective view of another embodiment of a pulley assembly to be incorporated into the surgical robotic arm of FIG. 1;

FIG. 8 is a perspective view of a semi-circular body of the pulley assembly of FIG. 7; and

FIG. 9 is a side view of the pulley assembly of FIG. 7, with parts removed.

### DETAILED DESCRIPTION

Embodiments of the presently disclosed surgical robotic arm are described in detail with reference to the drawings, in which like reference numerals designate identical or corresponding elements in each of the several views. As used herein the term “distal” refers to that portion of the robotic surgical system or component thereof, that is closer to a patient, while the term “proximal” refers to that portion of the robotic surgical system or component thereof, that is further from the patient.

As will be described in detail below, provided is a surgical robotic arm. The surgical robotic arm has a plurality of elongate members or links that are interconnected with one another and rotatable relative to one another via pulley systems while maintaining relative orientations of various links.

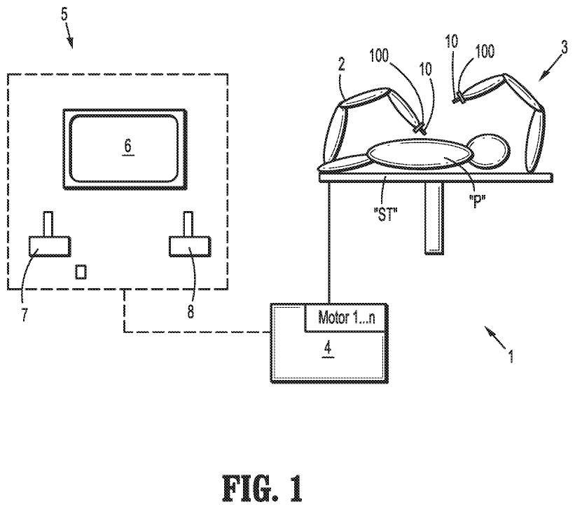

Referring initially to FIG. 1, a surgical system, such as, for example, a robotic surgical system 1, generally includes a plurality of surgical robotic arms 2, 3 having an instrument drive unit 100 and an electromechanical instrument 10 removably attached thereto; a control device 4; and an operating console 5 coupled with control device 4.

Operating console 5 includes a display device 6, which is set up in particular to display three-dimensional images; and manual input devices 7, 8, by means of which a person (not shown), for example a surgeon, is able to telemanipulate robotic arms 2, 3 in a first operating mode, as known in principle to a person skilled in the art. Each of the robotic arms 2, 3 may be composed of a plurality of members, which are connected through joints, as will be described in greater detail below. Robotic arms 2, 3 may be driven by electric drives (not shown) that are connected to control device 4. Control device 4 (e.g., a computer) is set up to activate the drives, in particular by means of a computer program, in such a way that robotic arms 2, 3, the attached instrument drive units 100, and thus electromechanical instrument 10 execute a desired movement according to a movement defined by means of manual input devices 7, 8. Control device 4 may also be set up in such a way that it regulates the movement of robotic arms 2, 3 and/or of the drives.

Robotic surgical system 1 is configured for use on a patient “P” lying on a surgical table “ST” to be treated in a minimally invasive manner by means of a surgical instrument, e.g., electromechanical instrument 10. Robotic surgical system 1 may also include more than two robotic arms 2, 3, the additional robotic arms likewise being connected to control device 4 and being telemanipulatable by means of operating console 5. A surgical instrument, for example, electromechanical surgical instrument 10, may also be attached to the additional robotic arm.

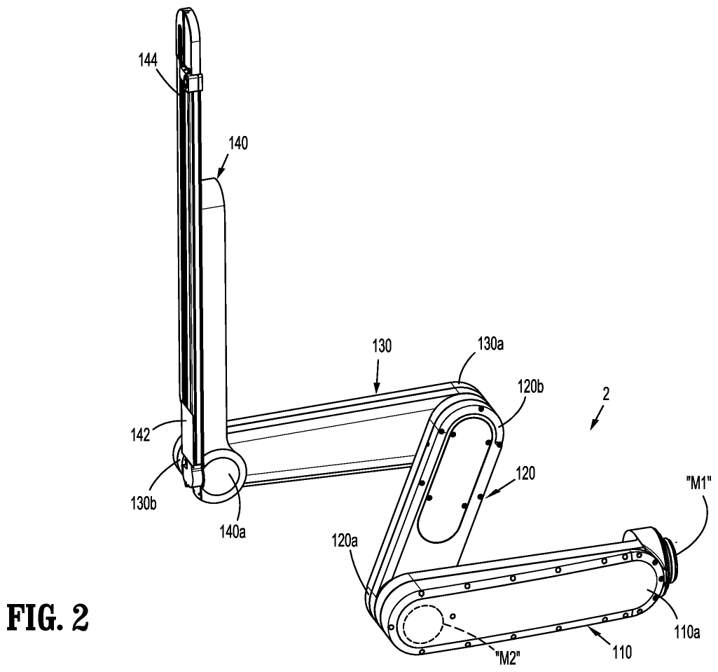

Control device 4 may control a plurality of motors, e.g., motors (Motor 1. . . n), with each motor configured to drive movement of robotic arms 2, 3 in a plurality of directions. Further, control device 4 may control a motor, such as, for example, a hollow core motor “M” (FIG. 2), configured to drive a relative rotation of elongate members of surgical robotic arm 2, as will be described in detail below.

For a detailed description of the construction and operation of a robotic surgical system, reference may be made to U.S. Patent Application Publication No. 2012/0116416, filed on Nov. 3, 2011, entitled “Medical Workstation,” the entire contents of which are incorporated by reference herein.

With reference to FIGS. 2-6B, surgical robotic arm 2 is configured to support surgical instrument 10 (FIG. 1) thereon and to selectively move surgical instrument 10 in a plurality of orientations relative to a small incision in a patient while maintaining surgical instrument 10 within the small incision. Robotic arm 2 includes a plurality of elongate members or links 110, 120, 130, 140 pivotably connected to one another to provide varying degrees of freedom to robotic arm 2. In particular, robotic arm 2 includes a first elongate member 110, a second elongate member 120, a third elongate member 130, and a fourth elongate member or link 140.

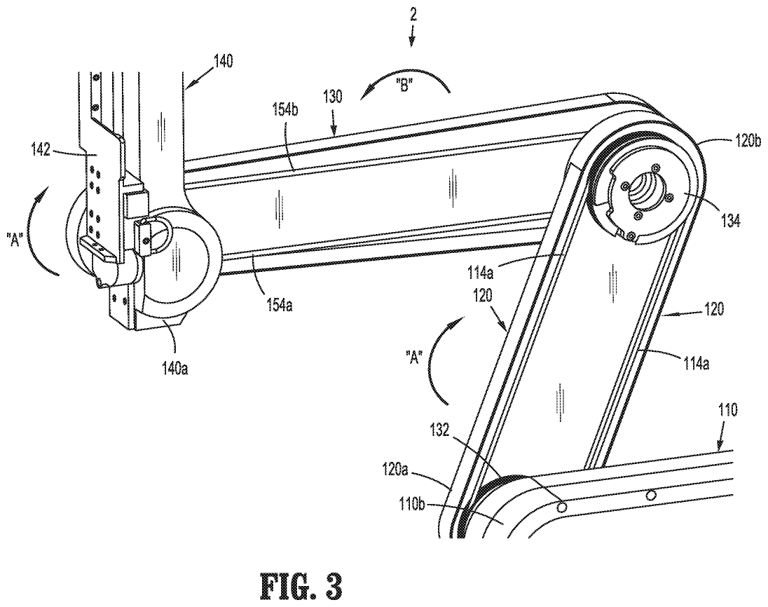

With reference to FIGS. 2 and 3, first elongate member 110 has a first end 110a and a second end 110b. First end 110a is configured to be rotatably coupled to a fixed surface, for example, a surgical cart, a surgical table, stanchion, operating room wall, or other surface present in the operating room. A first motor “M1” is operably coupled to first end 110a to rotate first elongate member 110 about a longitudinal axis thereof. Second end 110b of first elongate member 110 has a second motor, such as, for example, a hollow core motor “M2” attached thereto. Hollow core motor “M2” of first elongate member 110 is drivingly coupled to a first end of 120a of second elongate member 120 such that an actuation of motor “M2” effects a rotation of second elongate member 120 relative to first elongate member 110 about an axis defined through first end 110a of first elongate member 110 and second end 120a of second elongate member 120.

Second elongate member 120 includes a fixed pulley 132 disposed within first end 120a thereof. Fixed pulley 132 is fixedly connected to second end 110b of first elongate member 110 such that fixed pulley 132 of second elongate member 120 is non-rotatable relative to first elongate member 110. Second elongate member 120 has a second end 120b having a passive pulley 134 rotatably disposed therein. Passive pulley 134 of second elongate member 120 is operably coupled to fixed pulley 132 of second elongate member 120 via a pair of tethers or cables 114a, 114b. Passive pulley 134 of second elongate member 120 is also fixed to a first end 130a of third elongate member 130 such that rotation of passive pulley 134 of second elongate member 120 effects rotation of third elongate member 130 relative to second elongate member 120.

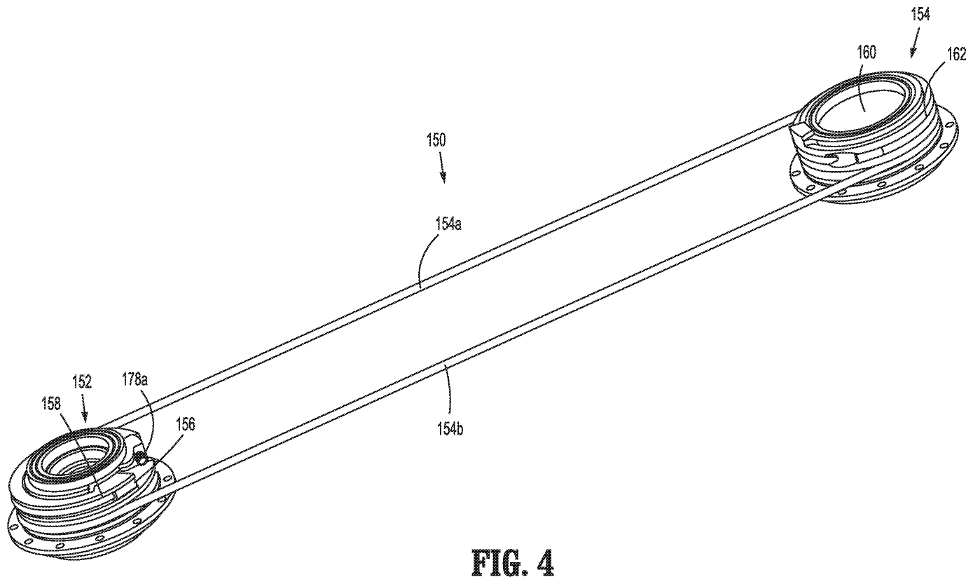

With reference to FIGS. 3 and 4, third elongate member 130 includes a fixed pulley 152 disposed within first end 130a thereof and non-rotatable relative to third elongate member 130. Third elongate member 130 has a second end 130b having a passive pulley 154 rotatably disposed therein. Passive pulley 154 of third elongate member 130 is operably coupled to fixed pulley 152 of third elongate member 130 via a pair of tethers or cables 154a, 154b. Cables 154a, 154b each have a first end 156 that is fixed within an annular groove 158 defined in fixed pulley 152, and a second end 160 that is fixed within an annular groove 162 defined in passive pulley 154 such that rotation of fixed pulley 152 effects rotation of passive pulley 154 in the same direction.

Cables 154a, 154b may be fabricated from steel or tungsten, or a composite of steel and tungsten, and may be comprised of a plurality of metal bands stacked on one another. In some embodiments, the portion of cables 154a, 154b fixed to pulleys 152, 154 may be tungsten whereas the remainder of cables 154a, 154b may be stainless steel. It is further contemplated that cables 154a, 154b may include an outer sheath, and an inner cable disposed within the sheath and having a greater stiffness compared to the sheath. The above-noted various embodiments of cables 154a, 154b are better than the prior art cables at reducing permanent and/or temporary stretching, and therefore have a longer lifespan.

Robotic arm 2 further includes an instrument carrier or rail 140. Passive pulley 154 of third elongate member 130 is non-rotatably coupled to a first end 140a of rail 140 such that rotation of passive pulley 154 of third elongate member 130 effects rotation of rail 140 relative to third elongate member 130. Instrument carrier or rail 140 has a slider 142 movably connected to a track or slide 144 of instrument carrier 140. Slider 142 moves, slides, or translates along a longitudinal axis defined by track 144 upon a selective actuation by motor(s) (not shown) supported on track 144 or motors (1 . . . n) of control device 4. As such, slider 142, with surgical instrument 10 (FIG. 1) connected thereto, can be moved to a selected position along track 144 of instrument carrier 140.

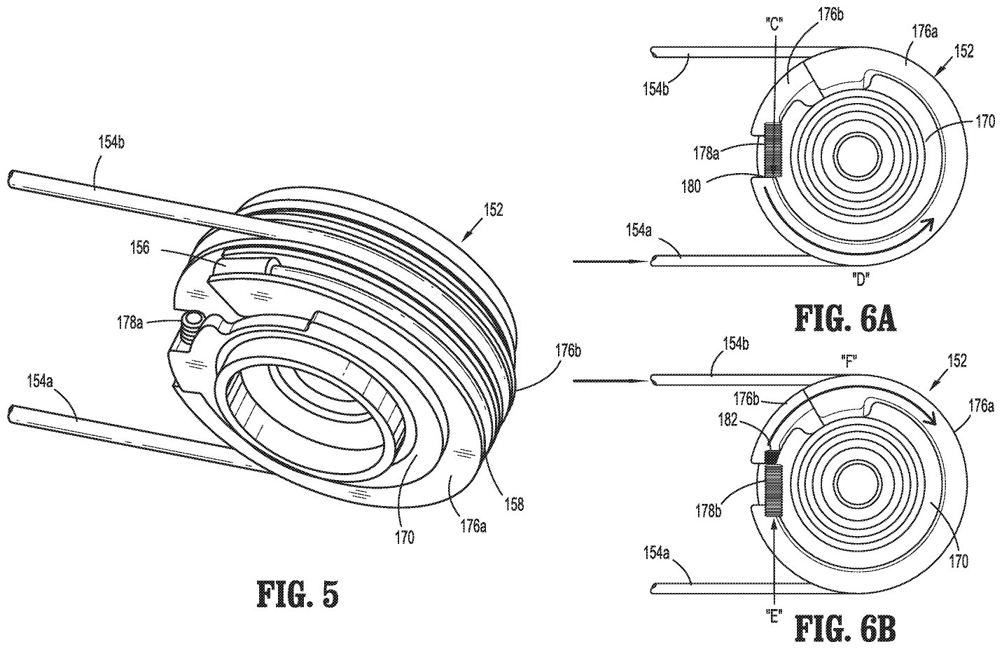

With reference to FIGS. 5, 6A, and 6B, fixed pulley 152 includes a hub 170, a first semi-circular element or body 176a, and a second semi-circular element or body 176b, together forming a pulley assembly 150 that allows a clinician to selectively change the tension of cables 154a, 154b to counteract the stretch that occurs in cables 154a, 154b over time. Hub 170 is disposed within first end 130a of third elongate member 130. First and second bodies 176a, 176b of pulley 152 are rotatably attached to hub 170. Bodies 176a, 176b each have a set screw 178a, 178b threadedly connected to hub 170 and having ends 180, 182 in engagement with bodies 176a, 176b.

A rotation of set screws 178a, 178b causes bodies 176a, 176b to rotate relative to hub 170 to change the tension in cables 154a, 154b, respectively. For example, a rotation of set screw 178a (e.g., in a clockwise direction) drives set screw 178a in a linear direction indicated by arrow “C” in FIG. 6A to linearly drive the end 180 of set screw 178a into hub 170. The linear movement of set screw 178a toward hub 170 drives a rotation of body 176a in the direction indicated by arrow “D” in FIG. 6A, thereby increasing the tension in cable 154a. A rotation of set screw 178b (e.g., in a clockwise direction) drives set screw 178b in a direction indicated by arrow “E” in FIG. 6B to linearly drive the end 182 of set screw 178b into hub 170. The linear movement of set screw 178b toward hub 170 drives a rotation of body 176b in the direction indicated by arrow “F” in FIG. 6B, thereby increasing the tension in cable 154a. To reduce tension in cables 154a, 154b, one or both of set screws 178a, 178b are rotated in an opposite direction of that required to increase tension.

In some embodiments, various fasteners or other mechanisms may be provided that cause bodies 176a, 176b to be selectively moved away from one another.

In operation, motor “M” of first elongate member 110 may be actuated, which rotates second elongate member 120 relative to first elongate member 110 in a first direction indicated by arrow “A” in FIG. 3 due to first end 120a of second elongate member 120 being operably coupled to motor “M2.” As second elongate member 120 rotates relative to first elongate member 110 in the first direction, passive pulley 134 of second elongate member 120 rotates in a second direction, indicated by arrow “B” in FIG. 3, which is opposite the first direction. Passive pulley 134 of second elongate member 120 rotates in the opposite direction as second elongate member 120 because fixed pulley 132 is non-rotatable relative to first elongate member 110. Since first end 130a of third elongate member 130 is operably coupled to passive pulley 134 of second elongate member 120, third elongate member 130 rotates with passive pulley 134 in the second direction the same amount as second elongate member 120 rotates in the first direction relative to first elongate member 110. In this way, the relative orientation of third elongate member 130 and first elongate member 110 remains constant during rotation of second elongate member 120.

As third elongate member 130 rotates in the second direction relative to second elongate member 120, passive pulley 154 of third elongate member 130 rotates in the first direction. Passive pulley 154 of third elongate member 130 rotates in the opposite direction as third elongate member 130 because fixed pulley 152 of third elongate member 130 is non-rotatable relative to third elongate member 130. Since first end 140a of slide 140 is operably coupled to passive pulley 154 of third elongate member 130, slide 140 rotates with passive pulley 154 in the first direction the same amount as third elongate member 130 rotates in the second direction relative to second elongate member 120. In this way, the relative orientation of second elongate member 120 and link 140 remains constant during rotation of third elongate member 130.

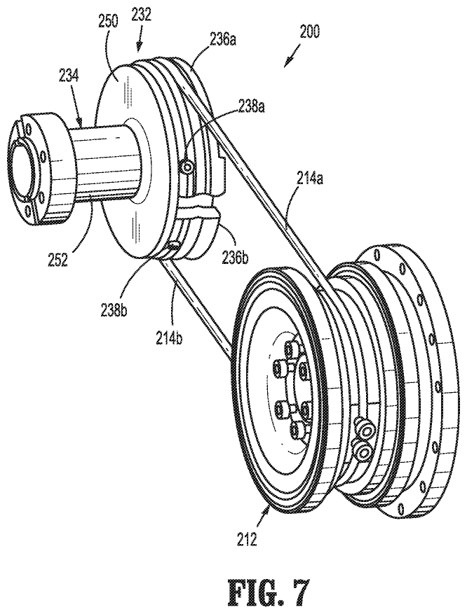

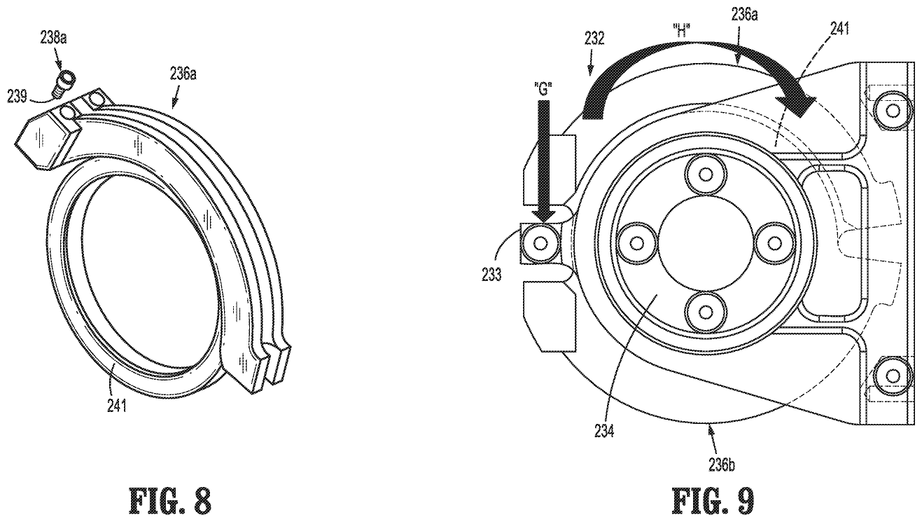

With reference to FIGS. 7-9, illustrated is another embodiment of a pulley assembly 200 to be incorporated into one of second or third elongate members 120, 130 (FIG. 2) of robotic arm 3. Pulley assembly 200 includes a first pulley 232, a second pulley 212, and a pair of tethers or cables 214a, 214b, similar to cables 154a, 154b described above. Pulley assembly 200 allows a clinician to selectively change the tension of cables 214a, 214b to counteract the stretch that occurs in cables 214a, 214b over time. First pulley 232 includes a hub or disc 234, a first semi-circular element or body 236a, and a second semi-circular element or body 236b. Hub 234 includes an annular flange member 250 and an axle 252 extending perpendicularly therefrom.

First and second bodies 236a, 236b of first pulley 232 each have a ring member 241, 243 that allow bodies 236a, 236b to rotatably interlock with one another. First and second bodies 236a, 236b of first pulley 232 are rotatably attached to annular flange member 250 of hub 234. Bodies 236a, 236b each have a set screw 238a, 238b threadedly connected to annular flange member 250 of hub 234 and having ends 239 in engagement with bodies 236a, 236b.

In operation, to account for a permanent stretching of cables 214a, 214b due to use over time, a tension in cables 214a, 214b may be adjusted. To adjust tension in cables 214a, 214b, one or both of set screws 238a, 238b of first pulley 232 may be rotated to drive screws 238a, 238b, in a linear direction indicated by arrow “G” in FIG. 9, to contact an end 239 of set screw 238a with a block 233 of annular flange member 250 of hub 234. Continued rotation of set screw 238a rotates first body 236a, in a direction indicated by arrow “H” in FIG. 9, away from second body 236b and block 233 to increase tension in cables 214a, 214b. Alternately or additionally, set screw 238b may be rotated to drive second body 236b away from first body 236a to increase tension in cables 214a, 214b. To reduce tension in cables 214a, 214b, one or both of set screws 238a, 238b are rotated in an opposite direction of that required to increase tension.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A robotic arm, comprising:
a first elongate member having a first end configured to be coupled to a fixed surface, and a second end;
a second elongate member having a first end rotatably connected to the second end of the first elongate member, and a second end;
a third elongate member having a first end rotatably connected to the second end of the second elongate member, and a second end configured to be coupled to a surgical instrument;
a motor attached to the second end of the first elongate member and configured to rotate the second elongate member relative to the first elongate member; and
a pulley assembly disposed within the second elongate member and including:
a first pulley disposed within the second end of the second elongate member;
a second pulley operably coupled to the first pulley via a first cable, the second pulley including:
a hub disposed within the first end of the second elongate member;
a first semicircular body rotatably attached to the hub; and
a first fastener movably coupled to the hub, wherein movement of the first fastener rotates the first semicircular body relative to the hub to change a tension in the first cable.

2. The robotic arm of claim 1, wherein the second pulley includes:
a second semicircular body rotatably attached to the hub of the second pulley; and
a second fastener movably coupled to the hub of the second pulley such that movement of the second fastener rotates the second semicircular body relative to the hub of the second pulley to change a tension in a second cable.

3. The robotic arm of claim 2, wherein the first and second semicircular bodies are disposed adjacent one another.

4. The robotic arm of claim 2, wherein the first cable has a first end fixed to the first semicircular body and a second end fixed to the first pulley, and the second cable has a first end fixed to the second semicircular body and a second end fixed to the first pulley.

5. The robotic arm of claim 1, wherein the first fastener is a screw that is threadingly coupled to the hub of the second pulley.

6. The robotic arm of claim 5, wherein the screw has an end in abutting engagement with the first semicircular body, wherein rotation of the screw drives the end of the screw toward the first semicircular body to rotate the first semicircular body.

7. The robotic arm of claim 1, wherein the first semicircular body has a first end and a second end and is disposed about the hub of the second pulley, the first fastener having an end in abutting engagement with the first end of the first semicircular body.

8. The robotic arm of claim 7, wherein the first cable has an end that is fixed to the second end of the first semicircular body.

9. The robotic arm of claim 1, wherein the first cable has a first end fixed to the first pulley and a second end fixed to the first semicircular body such that rotation of the first semicircular body relative to the hub of the second pulley increases the tension in the first cable.

10. A pulley assembly of a robotic arm, comprising:
a first cable having a first end and a second end;
a first pulley having the first end of the first cable fixed thereto;
a second pulley operably coupled to the first pulley via the first cable, the second pulley including:
a hub;
a first semicircular body rotatably attached to the hub and having the second end of the first cable fixed thereto; and
a first fastener movably coupled to the hub, wherein movement of the first fastener rotates the first semicircular body relative to the hub to change a tension in the first cable.

11. The pulley assembly of claim 10, wherein the second pulley includes:
a second semicircular body rotatably attached to the hub of the second pulley; and
a second fastener movably coupled to the hub of the second pulley such that movement of the second fastener rotates the second semicircular body relative to the hub of the second pulley to change a tension in a second cable.

12. The pulley assembly of claim 11, wherein the first cable has a first end fixed to the first semicircular body and a second end fixed to the first pulley, and the second cable has a first end fixed to the second semicircular body and a second end fixed to the first pulley.

13. The pulley assembly of claim 11, wherein the first and second semicircular bodies are disposed adjacent one another.

14. The pulley assembly of claim 10, wherein the first fastener is a screw that is threadingly coupled to the hub of the second pulley.

15. The pulley assembly of claim 14, wherein the screw has an end in abutting engagement with the first semicircular body, wherein rotation of the screw drives the end of the screw toward the first semicircular body to rotate the first semicircular body.

16. The pulley assembly of claim 10, wherein the first semicircular body has a first end and a second end and is disposed about the hub of the second pulley, the first fastener having an end in abutting engagement with the first end of the first semicircular body.

17. The pulley assembly of claim 16, wherein the first cable has an end that is fixed to the second end of the first semicircular body.

18. The pulley assembly of claim 10, wherein the first cable has a first end fixed to the first pulley and a second end fixed to the first semicircular body such that rotation of the first semicircular body relative to the hub of the second pulley increases the tension in the first cable.
